(19) United States
(12) Patent Application Publication
(10) Pub. No.: US 2006/0271043 A1
(43) Pub. Date: Nov. 30, 2006

(54) User-attached, manually operated fluid-driven arm lift device

(76) Inventor: Lupe Amy Gonzalez, Summit, NJ (US)

Correspondence Address: Kenneth Glynn, 24 Mine Street, Flemington, NJ, 08822, US

(21) Appl. No.: 11/139,420

(22) Filed: May 27, 2005

(51) Int. Cl. A61B 17/00

(52) U.S. Cl. 606/55; 601/5

(57) ABSTRACT

A user-attached, manually operated fluid-driven arm lift device for people who have difficulties in lifting an arm unassisted includes: a) a base support member adapted to fit about a portion of a side rib area of a human; b) a base support member attachment mechanism connected to the base support member and adapted to removably attach the base support member to the human; c) an arm support member having a top end and having a bottom end, and being hingedly connected to the base support member at its top end; d) a fluid-driven piston, cylinder and drive rod system connected to the base support member at the system bottom end and to the arm support member at system top end; e) a manually operated fluid pump and a fluid reservoir; and f) a fluid release mechanism.

### BACKGROUND OF THE INVENTION

1. Field of the Invention

The present invention relates to a device for lifting and lowering an arm, especially by raising and lowering the portion of a human arm above the elbow. More specifically, the present invention device offers a person with a need to have assistance in raising or holding an arm in an elevated position, without the need for an electric or battery operated device. Such a user of the present invention device could be someone who has an arm motive deficiency, such as someone who has or is suffering from an injury or disease, or it could be a healthy user who must hold an am in a raised position for an uncomfortably long period of time, such as an artist or certain types of workers. Thus, the present invention provides users the opportunity to raise, hold up and lower the arm without the need for batteries, motors and/or electronic controllers with power sources. The present invention device is attached to the torso and arm of the user and is activated manually, by cyclical movement of the hand, arm or combination thereof A simple release mechanism permits controlled lowering of the device arm component. The device is operated on the basis of movement of fluid by manually pumping, and the device may be fluid driven with a hydraulic system or with a pneumatic system.

2. Information Disclosure Statement

The following patents are representative of prior art of interest to the resent invention technology:

U.S. Pat. No. 6,709,411 describes a shoulder support for rehabilitation of musculo-skeletal shoulder disorders.

U.S. Pat. No. 5,203,763 describes a dynamic sling which provides a patient with therapeutic movement including joint approximation and proprioceptive input for an affected extremity joint, such as an arm and shoulder joint. The dynamic sling includes a first cuff for attachment to a patient's non-affected arm adjacent, or spaced from the shoulder along the humerus between the elbow joint and the shoulder joint. A second cuff is provided for attachment to the patient's affected arm adjacent the affected joint or spaced from the joint along the humerus between the elbow joint and the shoulder joint. An attachment member connects the first and second cuffs to one another crossing over the shoulder adjacent the affected joint. The attachment member connects adjacent to the rear of the non-affected arm and connects adjacent to the front of the affected arm. A guide member guides a portion of the attachment member between the first and second cuffs at a location generally contiguous with the cross over area of the attachment member over the shoulder of the affected arm to protect the patient against abrasions and bruising that may be caused by movement of the attachment member while transferring motion from the non-affected arm to the affected arm.

U.S. Pat. No. 4,896,660 describes the invention relating to an arm elevation support device comprising a generally monolithic humerus support, a contoured well shoulder anchor, and a radius and ulna support. The monolithic humerus support is operable to abut against a patient's side and underlies the humeral portion of a patient's arm. The contoured well shoulder anchor includes a contoured sleeve portion operable to be worn around the acromial portion of a patient's well arm and two straps which releasably connect the well shoulder anchor to the monolithic humerus support. The radius and ulna support connects to the monolithic structure and provides support for a patient's forearm and hand.

U.S. Pat. No. 4,651,719 describes a lightweight portable device which provides impart continuous passive motion to a user's shoulder. The device causes abduction and adduction and has the option of causing simultaneous rotation as well through use of a single actuator. The base of the device is curved to fit against a lateral portion of the user's torso and the device is held in position by a harness. An upper arm support is pivotally connected to and extends laterally from the base. A linear actuator extends between and is linked to the upper arm support and the base to cause abduction and adduction of the arm. A forearm support which is pivotally connected to the upper arm support and is also angularly adjustable relative to the upper arm support is linked to the base to cause rotation of the forearm support as the upper arm support is pivoted. The range of rotation can be varied or eliminated. The degree and speed of abduction and adduction can also be varied. The device is contained in a housing having a chamber with an extendable, two-part cover so that the operating mechanism is concealed.

U.S. Pat. No. 4,577,623 describes orthopedic equipment and more particularly a splint for mobilizing an upper limb, which comprises a vertical framework supporting, in its upper part, a brachial cradle articulated about a horizontal geometrical axis, intended substantially to coincide with the scapulo-humeral joint of the upper limb, and which is connected to the framework by a drive member capable of pivoting the cradle about said axis in movements of abduction/adduction with respect to the framework, said cradle being provided with an adjustable axial extension bearing a bent bar whose part substantially parallel to the extension supports an adjustable antebrachial sprint. The invention is applicable to the functional reeduction of the scapulo-humeral joint.

U.S. Pat. No. 4,487,199 describes a portable apparatus for imparting a continuous passive motion to a human joint. A support is provided for harnessing to parts of the body at one side of the joint and conveyor means movable relative to the support is provided for connection to a part at the other side of the joint, so that the latter forms a link which is flexed as the conveyor means moves back and forth relative to the support. Drive means insures that a substantially continuous slow rhythmic reciprocating movement is applied to the joint.

U.S. Pat. No. 3,976,057 describes a flexing apparatus including first and second straps to encircle the body above and below a joint, pivotable links between the straps to maintain alignment between them and piston and cylinder assemblies, extending between the straps. Adjustable bleed valves in the cyclinder ends provide calibrated leakage to present resistance to flexion and extension of the joint. An air supply and control are attached to the cylinder ends to provide controlled power flexion and extension of the joint for therapy, including a compressor, air filtering, regulating and lubricating devices, a two position solenoid operated valve and an electrical timing and switching circuit for energizing the solenoid.

U.S. Pat. No. 3,683,897 describes an apparatus including a member for attachment to a part of the body which member is capable of activating the part of the body cyclically. The member may comprise one or more double flexible sleeves alternatively or additionally it may comprise a mechanical linkage connected to a part of the body which includes linkage deforming means to cyclically deform the linkage and move the part of the body.

Notwithstanding the prior art, the present invention is neither taught nor rendered obvious thereby.

### SUMMARY OF THE INVENTION

The present invention is a user-attached, manually operated fluid-driven arm lift device for people who have difficulties in lifting an arm unassisted. It is primarily used for raising an arm by lifting the portion of the arm above the elbow, but could also be used to raise either the upper and lower arm or just the lower arm. It is particularly beneficial for people who have musculature or other range of motion problems from the shoulder joint down the arm. It is also particularly beneficial for people who have to hold an arm in a raised position to perform a task, such as an artist or worker. The present invention device includes: a) a base support member adapted to fit about a portion of a side rib area of a human; b) a base support member attachment means connected to the base support member and adapted to removably attach the base support member to the human; c) an arm support member having a top end and having a bottom end, and being hingedly connected to the base support member at its top end; d) a fluid-driven piston, cylinder and drive rod system having a movable piston within a cylinder, and having a drive rod connected top the piston, the system having a system top end at its drive rod and a system bottom end at its cylinder, and being connected to the base support member at the system bottom end and to the arm support member at system top end; e) a manually operated fluid pump and a fluid reservoir containing a fluid, the pump being functionally connected to the reservoir and the fluid reservoir being functionally connected to the fluid-driven piston, cylinder and drive rod system to enable a user to pump fluid to the cylinder to advance the fluid-driven piston and the drive rod; and f) a fluid release mechanism functionally connected to at least one of: (i) the fluid-driven piston, cylinder and drive rod system and (ii) the pump and fluid reservoir, the fluid release mechanism having at least a first position that prevents fluid from moving from the fluid-driven piston, cylinder and drive rod system to the reservoir, and a second position that permits fluid to move from the fluid-driven piston, cylinder and drive rod system to the reservoir.

When the present invention device is worn by a human and the manually operated pump is pumped, fluid is moved from the reservoir to the fluid-driven piston, cylinder and drive rod system, the piston and the drive rod are advanced to raise the arm support member and an arm of the human to which it is attached. When the fluid release mechanism is moved to its second position, fluid will flow from the cylinder to the reservoir to lower the support member and the arm of the human to which it is attached.

The term “fluid” as used herein is meant to be any liquid or gas or combination thereof that will translate movement of a pump to movement of a piston in a cylinder. Hence, the term includes, but is not limited to, pneumatic and hydraulic fluids. In some preferred embodiments of the present invention, the fluid is selected from the group consisting of an organic liquid, water, a non-toxic gas and air.

In some preferred embodiments of the present invention, the user-attached, manually operated fluid-driven arm lift device arm support member is an elongated, rigid concave member having an arm cushion therein. In some preferred embodiments of the present invention, the aforesaid arm cushion is a gel-containing cushion. Alternatively, the cushion could be a foam cushion, such as a urethane foam or other foam cushion, or it could be the living foam material referred to as space age mattress foam, or natural materials or combinations of any or all of the foregoing. Also, in some preferred embodiments of the present invention, the base support member is an elongated, rigid concave member having a torso cushion therein. This torso cushion I may preferably be a gel-containing cushion, or any of the foregoing other padding materials used in the arm support member.

The present invention device base support member attachment means is one that will perform two functions, namely, first, removably attach to a body to generally stay in place until a user desires to remove it, and, second, hold the base support member against the side of the user under the armpit and against the side of the rib cage so that when the device is operated, the piston, rod and arm support will lift in a consistent and correct orientation. In some preferred embodiments, the attachment means may be selected from the group consisting of an item of apparel having at least a torso section with said base support member attached thereto, a vest, at least one cross shoulder strap, and a torso harness. Instead of a vest, it could be a jacket, or other apparel. In some instances, it may be worm under other clothing, such as a loosely fitted or custom fitted jacket or other garment.

In many preferred embodiments of the present invention, the reservoir is located at the pump and the reservoir includes at least one connection tube connecting the reservoir to the cylinder. However, alternative arrangements could be used without exceeding the scope of the present invention. For example, the reservoir could be adjacent or within the cylinder, or the reservoir could be connected to the pump and the pump in turn connected to the cylinder. Thus, the pump could pull the fluid from the reservoir to the cylinder, or push it from the reservoir to the cylinder. Further, pumps may inherently operate as one way valves or there may be a separate one way valve permitting the fluid to flow to the cylinder, but not out of it unless intentionally released. Thus, the present invent also includes the release mechanism.

The present invention user-attached, manually operated fluid-driven arm lift device fluid release mechanism may be an on/off type mechanism or one that may be gradually moved from closed to various degrees of partial openings to fully open, such as a rotating knob or slider button. The valve or other release mechanism may be in line with the pump and cylinder or may be on a bypass line. In some embodiments, the present invention release mechanism is a valve having a one way flow function when in its first position and having a two way flow function when in its second position.

The pump may be any pump that will function to move the fluid to the cylinder. One type is the bulb pump similar to that which is used by nurses and doctors on blood pressure tourniquets, another is a cylinder-shaped pump that could be attached to the body and operated like a bellows. It could be attached or pressed against the body, e.g., at he waist or higher, so that it could be pressed to pump by moving the lower arm and/or hand reciprocally against it, or it could be located below the armpit area and pressed by the upper arm and/or elbow. In some embodiments, the pump is selected from a hand-held pump, a body mounted squeeze pump and a convertible pump having body mounted squeeze pump and hand-held pump capabilities.

While some preferred embodiments include the reservoir as an integral part of the device, in other embodiments, the device could obtain its fluid from a separate source, e. g. a detachable supply tank or even air take from the atmosphere. Thus, alternative embodiments of the present invention device include:

a) a base support member adapted to fit about a portion of a side rib area of a human;

b) a base support member attachment means connected to the base support member and adapted to removably attach the base support member to the human;

c) an arm support member having a top end and having a bottom end, and being hingedly connected to the base support member at its top end;

d) a fluid-driven piston, cylinder and drive rod system having a movable piston within a cylinder, and having a drive rod connected top the piston, the system having a system top end at its drive rod and a system bottom end at its cylinder, and being connected to the base support member at the system bottom end and to the arm support member at system top end;

e) a manually operated fluid pump and a fluid intake mechanism, the pump being functionally connected to the fluid intake mechanism and being functionally connected to the fluid-driven piston, cylinder and drive rod system to enable a user to pump fluid to the cylinder to advance the fluid-driven piston and the drive rod; and,

f) a fluid release mechanism functionally connected to at least one of: (i) the fluid-driven piston, cylinder and drive rod system and (ii) the pump and fluid intake mechanism, the fluid release mechanism having at least a first position that prevents fluid from moving from the fluid-driven piston, cylinder and drive rod system to fluid intake mechanism, and a second position that permits fluid to move from the fluid-driven piston, cylinder and drive rod system and out of the device;

wherein, when the arm support member is worn by a human and the manually operated pump is pumped, fluid is moved from the reservoir to the fluid-driven piston, cylinder and drive rod system, the piston and the drive rod are advanced to raise the arm support member and an arm of the human to which it is attached, and, when the fluid release mechanism is moved to its second position, fluid will flow from the cylinder out of the device to lower the support member and the arm of the human to which it is attached. The various details of the components may be as described above in conjunction with the embodiments having reservoirs, except that an open intake to the ambient air may be included or an intake connectable to another fluid source may be included.

### BRIEF DESCRIPTION OF THE DRAWINGS

The present invention should be more fully understood when the specification herein is taken in conjunction with the drawings appended hereto wherein:

FIG. 1 illustrates an oblique side view of one embodiment of a present invention user-attached, manually operated fluid-driven arm lift device for people who have difficulties in lifting an arm unassisted;

FIG. 2 illustrates a side view of another present invention user-attached, manually operated fluid-driven arm lift device in the lower position;

FIG. 3 illustrates a side view of the present invention user-attached, manually operated fluid-driven arm lift device shown in FIG. 2, but in the raised, upper position;

FIG. 4 illustrates a side view of another present invention user-attached, manually operated fluid-driven arm lift device in the lower position, with plural telescoping cylinder and piston mechanisms for significant extension;

FIG. 5 illustrates a side view of the FIG. 4 present invention user-attached, manually operated fluid-driven arm lift device in the raised position; and,

FIG. 6 illustrates a front view of another present invention embodiment with an apparel-type support means for the functional components of the device.

### DETAILED DESCRIPTION OF THE PRESENT INVENTION

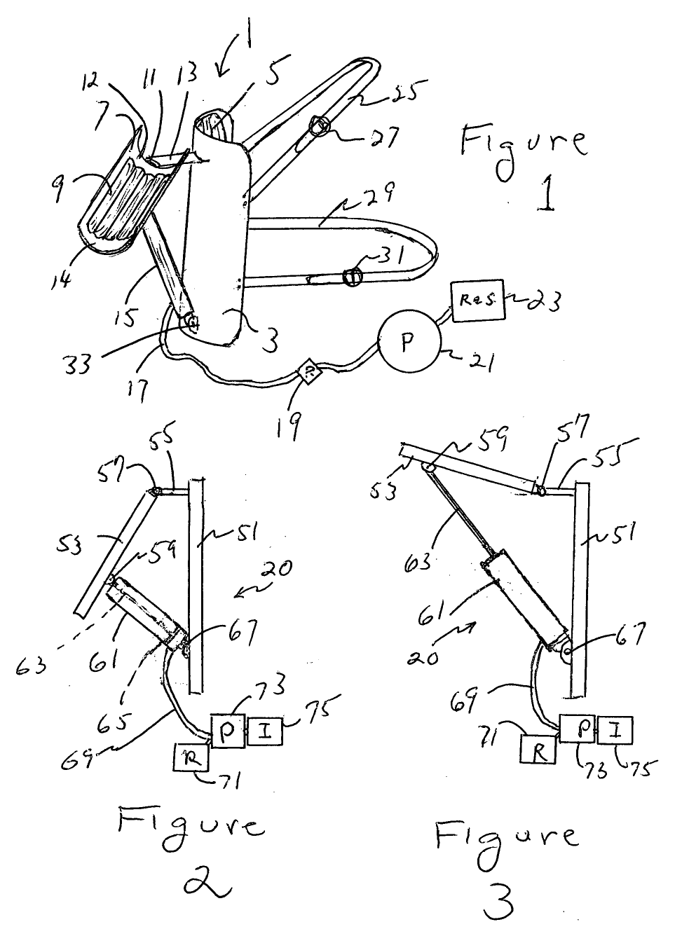

FIG. 1 illustrates an oblique side view of one embodiment of a present invention user-attached, manually operated fluid-driven arm lift device 1 for people who have difficulties in lifting an arm. Present invention arm lift device 1 includes a base support member 3 that is adapted to fit about a portion of a side rib area of a human. Base support member 3 is curved inwardly toward the body of a user so as to generally fit around the side rib cage and can be custom sized, molded and shaped to fit an individual body. It has inside padding, such as gel pads 5, to make the device more comfortable for the user.

The present invention arm lift device 1 also has an arm support member 7 designed to nest an upper arm area therein. In the position shown in FIG. 1, it would be for a right arm of a user between the armpit and the elbow. (The proportions and sizes are not necessarily to scale in the drawings and the details of the actual dimensions, connective positions and angles would vary for each user and, once the invention is taught, the fittage for each user would be within the skill of the artisan.) The arm support member 7 is concave in this embodiment, but could have any ergonomically acceptable shape. It preferably includes padding 9, such as gel pads, foam pads or the like, for user comfort. Arm support member 7 has a top end 12 and has a bottom end 14. Arm support member 7 is hingedly connected to the base support member 3 at its top end 12. In this embodiment, base support member 3 has a fixed bracket 13 and this connects to arm support member 7 via hinge 11. Bottom end 14 may have a terminus above the elbow of the user or it alternatively could extend down to a portion or all of a forearm, or otherwise as the particular user may require.

There is a fluid-driven piston, cylinder and drive rod system 15 having a movable piston within a cylinder, and having a drive rod connected top said piston. These components are not shown in detail in this Figure, but are shown and described in detail below, and any of those described or encompassed herein could be used with this FIG. 1 embodiment. The system has a system top end at its drive rod (not shown) and a system bottom end 33 at its cylinder bottom, and is connected to the base support member 3 at the system bottom end 33 and to the arm support member 7 at the system top end.

There a manually operated fluid pump 21 and a fluid reservoir 23 containing a fluid, i.e. liquid(s), gas(es) or a combination thereof. The pump is functionally connected to the reservoir and the fluid reservoir and pump are functionally connected to the fluid-driven piston, cylinder and drive rod system to enable a user to pump fluid to the cylinder to advance said fluid-driven piston and said drive rod. For example, in this embodiment, the reservoir 23 is upstream from the pump 21 and the cylinder 15 is downstream from the pump 21, but they are all functionally connected so that the pump 21 can be hand squeezed or forearm squeezed to move the fluid from the reservoir 23 to the cylinder 15 to raid the arm support member 7. The present invention arm lift device also includes a fluid release mechanism 19 that is functionally connected to at least one of: (i) the fluid-driven piston, cylinder and drive rod system 15 and (ii) the pump 21 and/or the fluid reservoir 23. In this case, the release mechanism 19 is located on hose 19. It has a first position that prevents fluid from moving from the fluid-driven piston, cylinder and drive rod system 15 to the pump 21 and reservoir 23 but allows fluid to flow to the system 15, and a second position that permits fluid to move (by gravity and/or the pushing weight of the arm and device) from the fluid-driven piston, cylinder and drive rod system 15 to the reservoir 23.

There is a base support member attachment means connected to the base support member and adapted to removably attach the base support member to the user. In this example, the attachment means is an upper shoulder strap 25 with adjustment buckle 27 and a waist belt strap 29 with buckle 31. Other types could be used in place of those shown. For example, straps for each shoulder and plural horizontal straps could be used. They could be wide straps of cloth or canvas, leather or webbing such as nylon or seatbelt material. The adjusting and closing mechanism could be snaps, zippers, hook and loop, sometimes called Velcro, buckle, of any other closure mechanism.

When the present invention device 1 is worn by a human and the manually operated pump 21 is pumped, fluid is moved from the reservoir 23 to the fluid-driven piston, cylinder and drive rod system 15, and the piston and drive rod are advanced to raise the arm support member 7 and an arm of the human to which it is attached. Obversely, when the fluid release mechanism 19 is moved to its second position, fluid will flow from the cylinder to the reservoir to lower the arm support member 7 and the arm of the human to which it is attached.

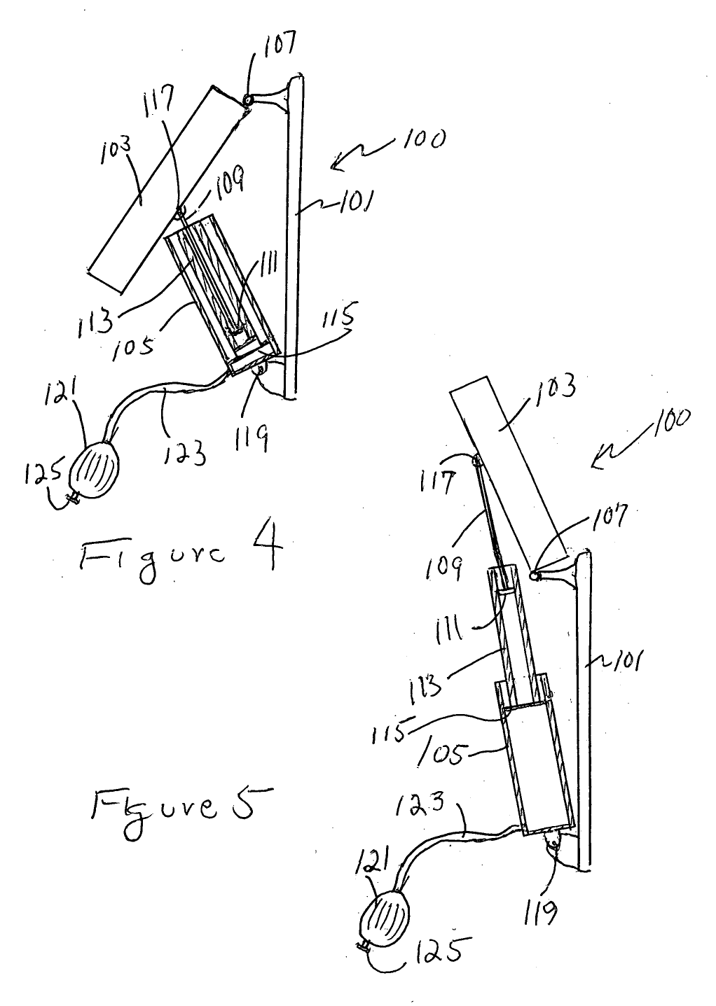

FIG. 2 illustrates a side view of another present invention user-attached, manually operated fluid-driven arm lift device 20 in the lower position, and FIG. 3 illustrates a side view of the present invention user-attached, manually operated fluid-driven arm lift device 20 shown in FIG. 2, but in the raised, upper position. Both Figures are described here collectively. Device 20 has a base support member 51 and an arm support member 53 rotatably connected to one another at bracket 55 with hinge 57, as shown. Drive rod 63 is rotatably connected to arm support member 53 at bolt and bracket 59 and cylinder 61 is rotatably connected to base support member 51 at bolt and bracket 67. Piston 65 and drive rod 63 are moved by air or other fluid moving in and out of cylinder 61 in a manner similar to that described in conjunction with FIG. 1 above, except that no reservoir is included. Instead, there is a fluid intake valve 75 that permits intake but prevents outflow of fluid when pump 73 is pumped. Release 71 is opened to release air or other fluid from cylinder 61 to lower the arm support member 53. Release 71 is closed in FIG. 3 and the pump 73 has been pumped to add fluid to cylinder 61, move the piston and drive rod 63 to raise support member 53, as shown.

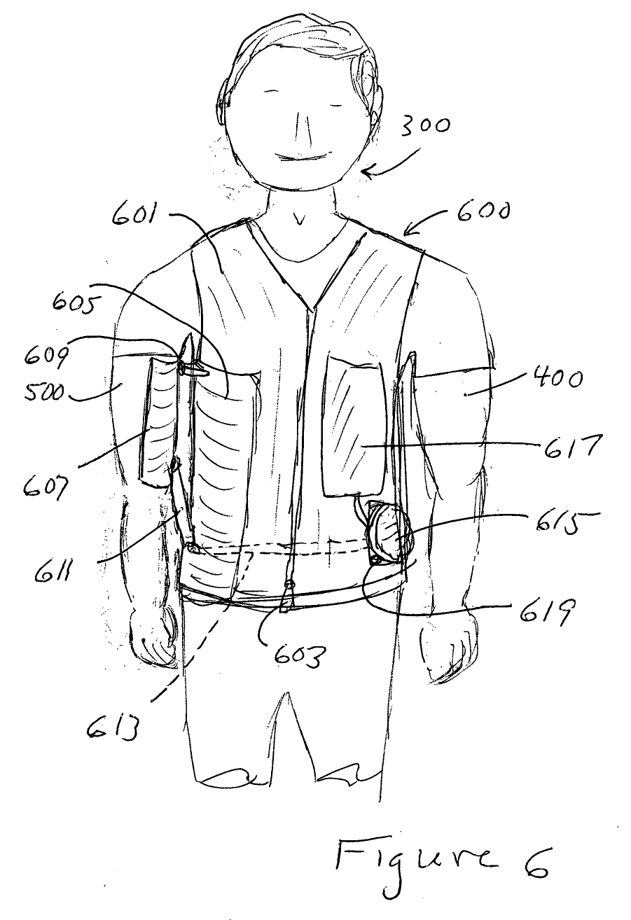

FIG. 4 illustrates a side view of another present invention user-attached, manually operated fluid-driven arm lift device 100 in the lower position, and FIG. 5 illustrates a side view of the FIG. 4 arm lift device 100 in the raised position. This embodiment has plural telescoping cylinder and piston mechanisms for significant extension. Both Figures are described here collectively.

Device 100 has a base support member 101 and an arm support member 103, both being padded adequately and curved or otherwise designed to fit the perspective body areas as described above. They are rotatably connected to one another with hinge 107, as shown. Drive rod 109 is rotatably connected to arm support member 103 at bolt and bracket 117 and cylinder 105 is rotatably connected to base support member 101 at bolt and bracket 119. Within cylinder 105 is a second cylinder 133. Second cylinder 113 has a drive rod 109 and piston 111, as shown. Second cylinder 113 has a base 115 that has a hole in it to permit fluid to drive piston 111. However, base 115 also acts as a piston once rod 109 is extended, as then additional fluid entering cylinder 105 will drive up base 115, and the cylinder 113, as well as the already extended piston 11 and rod 109. Thus, the pistons, second cylinder and drive rod are moved by air or other fluid moving in and out in a manner similar to that used with a nurse's manually activated and screw released hand pump; namely, here, pump 121 and gradual release screw 125. The pump is connected to main cylinder 105 via hose 123. The pump may be hand operated described or removably connected to an attachment means for forearm pumping against the body. Release screw 125 acts as a fluid intake valve that permits intake but prevents outflow of fluid when pump 121 is pumped, and is opened to release air or other fluid from the telescoping cylinders to lower the arm support member 103.

FIG. 6 illustrates a front view of another present invention device 600 with an apparel-type support means for the functional components of the device. User 300 has a good arm, left arm 400, and an injured arm, right arm 500 that cannot be raised on its own. The injury is at the shoulder area, but the hand and elbow are functional. Present invention device 600 includes vest 601, with any available closure means, such as zipper 603. In fact, the vest could be a jacket, slip-over or other apparel that would support the functional components of the device. There is a base support member 605 and an arm support member 607 rotatably connected to one another at hinge 609, as shown. There is a drive rod and piston(not shown) and cylinder 611, similar to the ones shown in FIG. 2, that are rotatably connected to arm support member 607 and base support member 605. The piston and drive rod are moved by air or other fluid moving in and out of cylinder 611 in a manner similar to that described in conjunction with FIG. 1 above. Fluid line 613 connects the cylinder 611 to pump 615, with release mechanism 619. Pump 615 is forearm operated and is connected to the fluid reservoir 617, built into the vest 601, as shown. This device 600 operates in the same fashion as the devices described above.

Obviously, numerous modifications and variations of the present invention are possible in light of the above teachings. It is therefore understood that within the scope of the appended claims, the invention may be practiced otherwise than as specifically described herein.

What is claimed is:

1. A user-attached, manually operated fluid-driven arm lift device, which comprises:
a) a base support member adapted to fit about a portion of a side rib area of a human;
b) a base support member attachment means connected to said base support member and adapted to removably attach said base support member to said human;
c) an arm support member having a top end and having a bottom end, and being hingedly connected to said base support member at its top end;
d) a fluid-driven piston, cylinder and drive rod system having a movable piston within a cylinder, and having a drive rod connected top said piston, said system having a system top end at its drive rod and a system bottom end at its cylinder, and being connected to said base support member at said system bottom end and to said arm support member at system top end;
e) a manually operated fluid pump and a fluid reservoir containing a fluid, said pump being functionally connected to said reservoir and said fluid reservoir being functionally connected to said fluid-driven piston, cylinder and drive rod system to enable a user to pump fluid to said cylinder to advance said fluid-driven piston and said drive rod; and,
f) a fluid release mechanism functionally connected to at least one of: (i) said fluid-driven piston, cylinder and drive rod system and (ii) said pump and fluid reservoir, said fluid release mechanism having at least a first position that prevents fluid from moving from said fluid-driven piston, cylinder and drive rod system to said reservoir, and a second position that permits fluid to move from said fluid-driven piston, cylinder and drive rod system to said reservoir;
wherein, when said device is worn by a human and said manually operated pump is pumped, fluid is moved from said reservoir to said fluid-driven piston, cylinder and drive rod system, said piston and said drive rod are advanced to raise said arm support member and an arm of said human to which it is attached, and, when said fluid release mechanism is moved to its second position, fluid will flow from said cylinder to said reservoir to lower said support member and said arm of said human to which it is attached.

2. The user-attached, manually operated fluid-driven arm lift device of claim 1 wherein said arm support member is an elongated, rigid concave member having an arm cushion therein.

3. The user-attached, manually operated fluid-driven arm lift device of claim 1 wherein said base support member is an elongated, rigid concave member having a torso cushion therein.

4. The user-attached, manually operated fluid-driven arm lift device of claim 2 wherein said arm cushion is a gel-containing cushion.

5. The user-attached, manually operated fluid-driven arm lift device of claim 3 wherein said torso cushion is a gel-containing cushion.

6. The user-attached, manually operated fluid-driven arm lift device of claim 1 wherein said fluid is selected from the group consisting of an organic liquid, water, a non-toxic gas and air.

7. The user-attached, manually operated fluid-driven arm lift device of claim 1 wherein said base support member attachment means is selected from the group consisting of an item of apparel having at least a torso section with said base support member attached thereto, a vest, at least one cross shoulder strap, and a torso harness.

8. The user-attached, manually operated fluid-driven arm lift device of claim 1 wherein said reservoir includes at least one connection tube connecting said reservoir to said cylinder.

9. The user-attached, manually operated fluid-driven arm lift device of claim 1 wherein said fluid release mechanism is a valve having a one way flow function when in its first position and having a two flow function when in its second position.

10. The user-attached, manually operated fluid-driven arm lift device of claim 1 wherein said pump is selected from a hand-held pump, a body mounted squeeze pump and a convertible pump having body mounted squeeze pump and hand-held pump capabilities.

11. A user-attached, manually operated fluid-driven arm lift device, which comprises:
a) a base support member adapted to fit about a portion of a side rib area of a human;
b) a base support member attachment means connected to said base support member and adapted to removably attach said base support member to said human;
c) an arm support member having a top end and having a bottom end, and being hingedly connected to said base support member at its top end;
d) a fluid-driven piston, cylinder and drive rod system having a movable piston within a cylinder, and having a drive rod connected top said piston, said system having a system top end at its drive rod and a system bottom end at its cylinder, and being connected to said base support member at said system bottom end and to said arm support member at system top end;
e) a manually operated fluid pump and a fluid intake mechanism, said pump being functionally connected to said fluid intake mechanism and being functionally connected to said fluid-driven piston, cylinder and drive rod system to enable a user to pump fluid to said cylinder to advance said fluid-driven piston and said drive rod; and,
f) a fluid release mechanism functionally connected to at least one of: (i) said fluid-driven piston, cylinder and drive rod system and (ii) said pump and fluid intake mechanism, said fluid release mechanism having at least a first position that prevents fluid from moving from said fluid-driven piston, cylinder and drive rod system to fluid intake mechanism, and a second position that permits fluid to move from said fluid-driven piston, cylinder and drive rod system and out of said device;
wherein, when said device is worn by a human and said manually operated pump is pumped, fluid is moved from said reservoir to said fluid-driven piston, cylinder and drive rod system, said piston and said drive rod are advanced to raise said arm support member and an arm of said human to which it is attached, and, when said fluid release mechanism is moved to its second position, fluid will flow from said cylinder out of said device to lower said support member and said arm of said human to which it is attached.

12. The user-attached, manually operated fluid-driven arm lift device of claim 11 wherein said arm support member is an elongated, rigid concave member having an arm cushion therein.

13. The user-attached, manually operated fluid-driven arm lift device of claim 11 wherein said base support member is an elongated, rigid concave member having a torso cushion therein.

14. The user-attached, manually operated fluid-driven arm lift device of claim 12 wherein said arm cushion is a gel-containing cushion.

15. The user-attached, manually operated fluid-driven arm lift device of claim 13 wherein said torso cushion is a gel-containing cushion.

16. The user-attached, manually operated fluid-driven arm lift device of claim 11 wherein said fluid is selected from the group consisting of an organic liquid, water, a non-toxic gas and air.

17. The user-attached, manually operated fluid-driven arm lift device of claim 11 wherein base support member attachment means is selected from the group consisting of an item of apparel having at least a torso section with said base support member attached thereto, a vest, at least one cross shoulder strap, and a torso harness.

18. The user-attached, manually operated fluid-driven arm lift device of claim 11 wherein said pump includes at least one connection tube connecting said pump to said cylinder.

19. The user-attached, manually operated fluid-driven arm lift device of claim 11 wherein said fluid release mechanism is a valve having a one way flow function when in its first position and having a two flow function when in its second position.

20. The user-attached, manually operated fluid-driven arm lift device of claim 11 wherein said pump is selected from a hand-held pump, a body mounted squeeze pump and a convertible pump having body mounted squeeze pump and hand-held pump capabilities.
